(19) United States
(12) Patent Application Publication
(10) Pub. No.: US 2026/0166320 A1
(43) Pub. Date: Jun. 18, 2026

(54) SYSTEM AND METHOD FOR OPTICAL STIMULATION OF A RETINAL IMPLANT

(71) Applicant: Science Corporation, Alameda, CA (US)

(72) Inventors: Russell Sharpe, Alameda, CA (US); Marat Rostov, Alameda, CA (US); Michael Schaub, Alameda, CA (US); Elizabeth Carroll, Alameda, CA (US); Christina Skieller, Alameda, CA (US); Wei Cui, Alameda, CA (US); Scott Kottmer, Alameda, CA (US); Seton Schiraga, Alameda, CA (US)

(73) Assignee: Science Corporation, Alameda, CA (US)

(21) Appl. No.: 19/408,031

(22) Filed: Dec. 3, 2025

Related U.S. Application Data
(60) Provisional application No. 63/733,541, filed on Dec. 13, 2024.
(60) Provisional application No. 63/768,414, filed on Mar. 7, 2025.

(51) Int. Cl. A61N 1/36; G02B 27/00; G02B 27/01

(52) U.S. Cl. CPC A61N 1/36046; A61N 1/36132; A61N 1/36139; G02B 27/0093; G02B 27/0172; G02B 27/0176; G02B 2027/0138; G02B 2027/014; G02B 2027/0154; G02B 2027/0178; G02B 27/0179; G02B 2027/0181

(57) ABSTRACT

The system can include: a light engine, an eye sensor, and a processing system. However, the method can additionally or alternatively include any other suitable components. In variants, the system can function to project a time series of patterned light frames onto the retina of an eye. In a specific example, the system can function to optically stimulate a retinal implant, wherein the retinal implant stimulates (e.g., activates) retinal cells.

### CROSS-REFERENCE TO RELATED APPLICATIONS

This application claims the benefit of U.S. Provisional Application No. 63/768,414 filed 07 Mar. 2025, and U.S. Provisional Application No. 63/733,541 filed 13 Dec. 2024, each of which is incorporated in its entirety by this reference.

### TECHNICAL FIELD

This invention relates generally to the optics field, and more specifically to a new and useful system and method for optical stimulation in the optics field.

### BRIEF DESCRIPTION OF THE SEVERAL VIEWS OF THE DRAWINGS

FIG. 1 is a schematic representation of a variant of the system.

FIG. 2 is a schematic representation of a variant of the method.

FIG. 3A is a schematic representation of a first example of the system, wherein a set of light sources produces unpatterned light.

FIG. 3B is a schematic representation of a second example of the system, wherein a set of light sources produces patterned light.

FIGS. 4A and 4B depict an example of a light engine dynamically illuminating a sub-field of view (e.g., a portion of the field of view that is aligned with a retinal implant), where a sub-region of the set of light sources produces unpatterned light.

FIGS. 5A and 5B depict an example of a light engine dynamically illuminating a sub-field of view (e.g., a portion of the field of view that is aligned with a retinal implant), where a sub-region of the set of light sources produces patterned light.

FIG. 6A is a schematic representation of a first example of the method.

FIG. 6B is a schematic representation of a first example of the method.

FIG. 7 depicts a specific example of the system.

FIG. 8 depicts a specific example of optical components of the system.

FIG. 9 depicts a specific example of a light engine.

FIG. 10 depicts a specific example of a combiner lens.

FIGS. 11 and 12 depict illustrative examples of the system.

FIGS. 13A and 13B depict illustrative examples of the system, including a light engine mounted to the temple of a glasses frame.

FIG. 14 depicts another specific example of optical components of the system.

FIG. 15 depicts a specific example of electronic components of the system.

FIG. 16 depicts a specific example of selective illumination of a sub-region of a light engine.

FIGS. 17A and 17B depict specific examples of the combiner lens.

FIG. 18 depicts a specific example of a retinal implant.

FIG. 19 depicts an illustrative example of gaze tracking data collected using an eye sensor.

### DETAILED DESCRIPTION OF THE INVENTION

The following description of the embodiments of the invention is not intended to limit the invention to these embodiments, but rather to enable any person skilled in the art to make and use this invention.

### 1. Overview

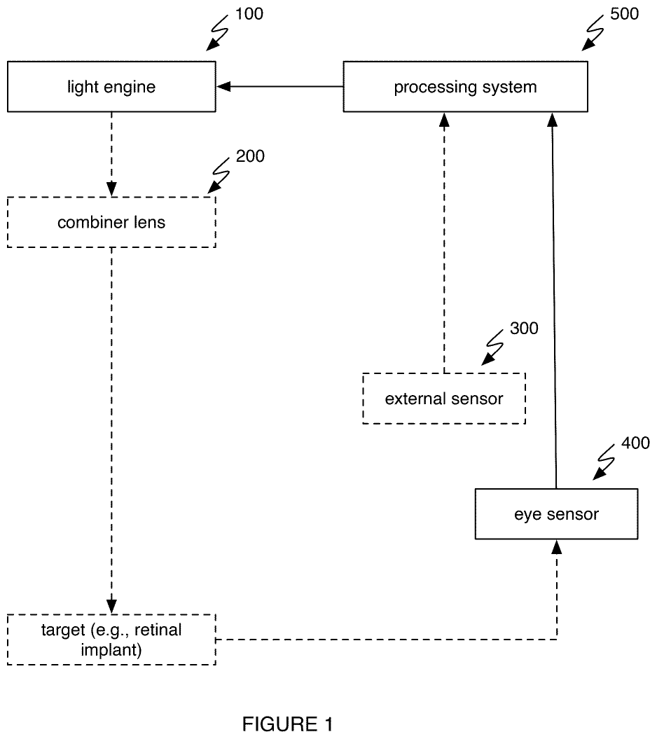

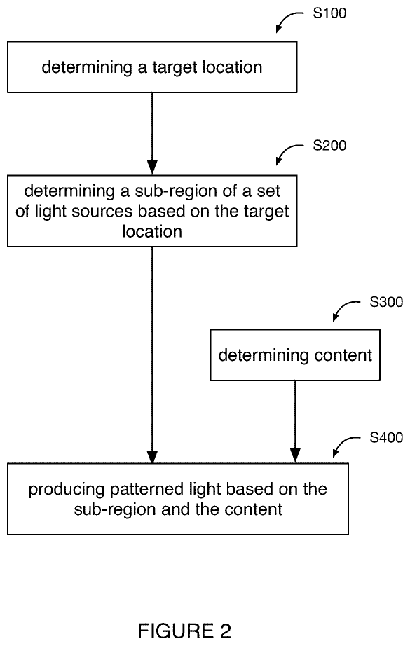

As shown in FIG. 1, the system can include: a light engine 100, an eye sensor 400, and a processing system 500. However, the system can additionally or alternatively include any other suitable components. As shown in FIG. 2, the method can include: determining a target location, determining a sub-region of a set of light sources based on the target location, determining content, and producing patterned light based on the sub-region and the content. However, the method can additionally or alternatively include any other suitable steps.

In variants, the system can function to project a time series of patterned light frames towards the retina of an eye (e.g., onto a retinal implant coupled to the retina of the eye). For example, the system can function to restore vision for users who have lost function of some or all of their photoreceptors (e.g., degenerated and/or otherwise impaired photoreceptors), ganglia, and/or other sensory cells. In an example, the system functions to optically stimulate a retinal implant, wherein the retinal implant stimulates (e.g., activates) retinal cells.

The system can optionally be or include a wearable projector system (e.g., glasses). For example, the wearable projector system can include a light engine, a combiner lens, an external sensor (e.g., camera), an eye sensor, and a processing system. In an example, the light engine can include an array of light sources (e.g., vertical-cavity surface-emitting lasers) and an optional display module 140 (e.g., spatial light modulator). The system can project a time series of patterned light frames (e.g., encoding content measured by the external sensor) towards a retina of a user. In a specific example, the patterned light frames can be projected onto a retinal implant, wherein resulting electrical signals generated by the retinal implant in response to receiving the patterned light can be delivered to native retinal cells and subsequently interpreted by the brain (e.g., such that the user can visualize the content). In an example, a sub-region of the set of light sources can be selectively illuminated (e.g., wherein the portion of light emitted by the sub-region is subsequently patterned via the spatial light modulator), projecting light to a sub-field of view that corresponds to the current retinal implant location. In a specific example, the selected sub-region can be iteratively updated based on the tracked gaze of the user (e.g., via the eye sensor). The position of the light engine and/or the combiner lens can optionally be adjustable to accommodate for different inter-pupillary distances between users.

However, the system/method can be otherwise performed.

### 2. Technical Advantages

Variants of the technology can confer one or more advantages over conventional technologies.

First, variants of the technology can restore and/or augment sight in users. In some diseases like retinitis age-related macular degeneration, the photoreceptors are damaged while the cells of the optic nerve, the retinal ganglion cells (RGCs) and/or retinal bipolar cells, remain functional. Variants of the technology can include a wearable projector system that emits patterned light to stimulate cells in the retina (e.g., via stimulation of a retinal implant) to restore visual input to the brain (e.g., replacing foveal vision). In a specific example, the wearable projector system can be lightweight (e.g., less than 180 g, less than 160 g, less than 150 g, less than 100 g, etc.). In another specific example, the wearable projector system can be fully contained within and/or on a glasses form factor, which can improve the appearance of the system for users and/or enable the system to operate without the use of fiber optic cables connecting optical components.

Second, variants of the wearable projector system and method can include dynamically illuminating selected sub-regions of an array of light sources (e.g., lasers) to project light onto a sub-field of view (sub-FOV) that corresponds to a current, tracked retinal implant location. In variants, this selective illumination can provide a larger eye box size while limiting the amount of projected light illuminating portions of the eye beyond the retinal implant. For example, the system can provide an expanded field of view (e.g., at least 20 degrees, at least 25 degrees, at least 30 degrees, etc.) and/or an expanded eye box size (e.g., at least 2 mm, at least 5 mm, at least 6 mm, at least 10 mm, etc.). In variants, this selective illumination can additionally or alternatively provide energy savings to the system (e.g., by reducing the number of light sources that are illuminated at a given time).

However, further advantages can be provided by the system and method disclosed herein.

### 3. System

As shown in FIG. 1, the system can include: a light engine 100, an eye sensor 400, and a processing system 500. The system can optionally include a combiner lens 200, an external sensor 300, a retinal implant 600, one or more optical components, one or more supplemental sensors, one or more user interface components, power source (e.g., a battery), and/or any other suitable elements.

In variants, the system can include and/or interface with systems as described in U.S. application Ser. No. 17/593,274 filed 14 Sep. 2021, U.S. application Ser. No. 19/210,450 filed 16 May 2025, U.S. application Ser. No. 18/796,038 filed 6 Aug. 2024, U.S. application Ser. No. 19/101,736 filed 6 Feb. 2025, U.S. application Ser. No. 19/143,639 filed 26 Jun. 2025, Ser. No. 19/044,388 filed 3 Feb. 2025, U.S. application Ser. No. 16/631,029 filed 14 Jan. 2020, U.S. application Ser. No. 17/296,685 filed 25 May 2021, U.S. application Ser. No. 19/188,842 filed 24 Apr. 2025, and/or U.S. application Ser. No. 18/740,854 filed 12 Jun. 2024, each of which is incorporated in its entirety by this reference.

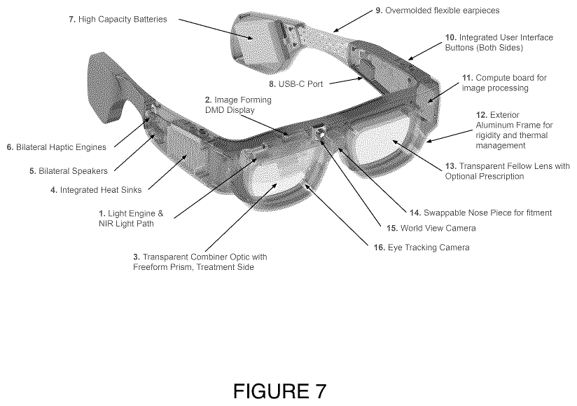

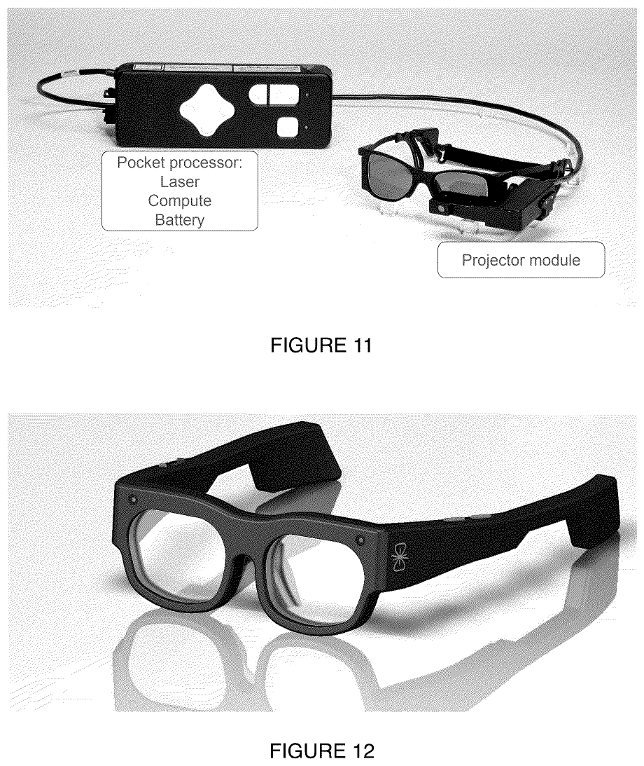

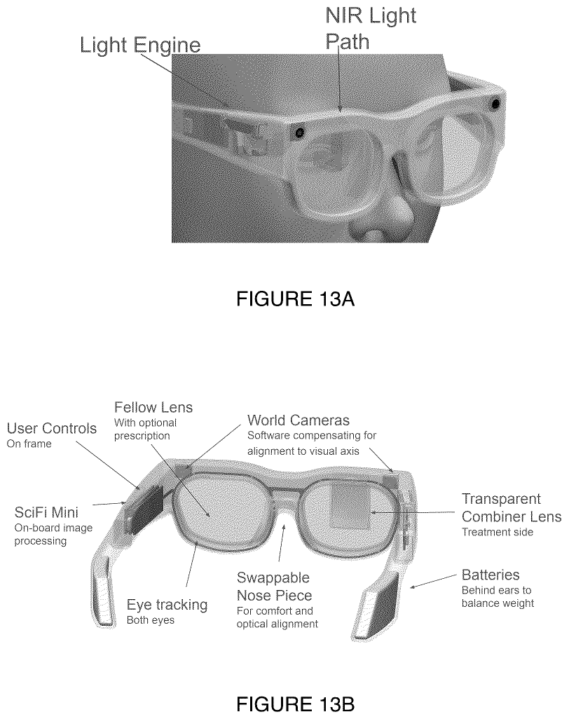

The system can optionally be or include a wearable system (e.g., a wearable projector system). In a specific example, the system can be or include glasses (e.g., glasses frame). Examples are shown in FIG. 7, FIG. 11, FIG. 12, FIG. 13A, and FIG. 13B. In a specific example, one or more of the light engine 100, the combiner lens 200, the external sensor 300, the eye sensor 400, and/or the processing system 500 are contained within and/or coupled to the glasses frame. The glasses can optionally be modular such that the system components can be arranged within or on the glasses for right eye light projection or left eye light projection. In an example, the material of the frame of the glasses can optionally include a metal (e.g., aluminum, copper, etc.). In a specific example, the material of the frame of the glasses can optionally have high thermal conductivity (e.g., at least 50 W/(m·K), at least 100 W/(m·K), at least 200 W/(m·K), etc.). The frame of the glasses can optionally include one or more internal heat sinks. The frame of the glasses can optionally include one or more rigid regions and/or one or more flexible regions (e.g., the temple, the temple tips, the nose pads, the bridge, etc.).

The system can project a time series of patterned light frames onto one or more targets. The target is preferably a retinal implant 600 or a component thereof, but can additionally or alternatively include one or more eyes or component(s) thereof (e.g., pupil, fundus, fovea, retina, a portion of a retina, retinal cells, etc.). For example, the target can include one or more pixels (e.g., photovoltaic cells, photodiodes, etc.) of the retinal implant 600. An output light path can define the optical path of output light from the combiner lens 200 to a target location. The target location can be the location of the target (e.g., the center of the target), a region encompassing the target (e.g., a region of the retina that includes the target), a location and/or region within the target (e.g., a target location on the retina, a target location on the retinal implant 600, etc.), a location of an eye feature, and/or any other location associated with the target and/or the eye. In a specific example, the target location can be the location of the retinal implant 600 (e.g., of the center of the retinal implant 600).

### 3.1. The Light Engine 100

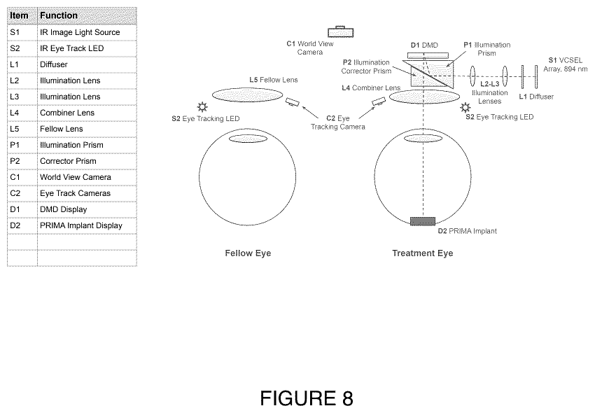

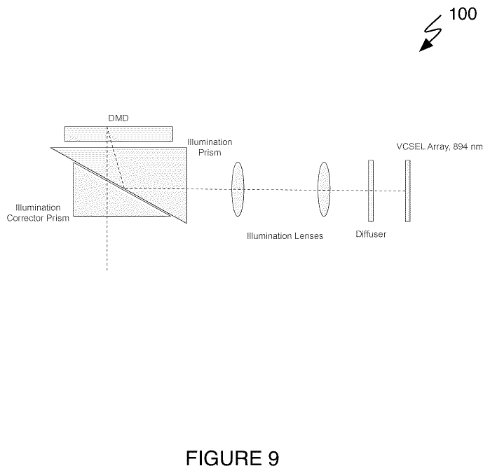

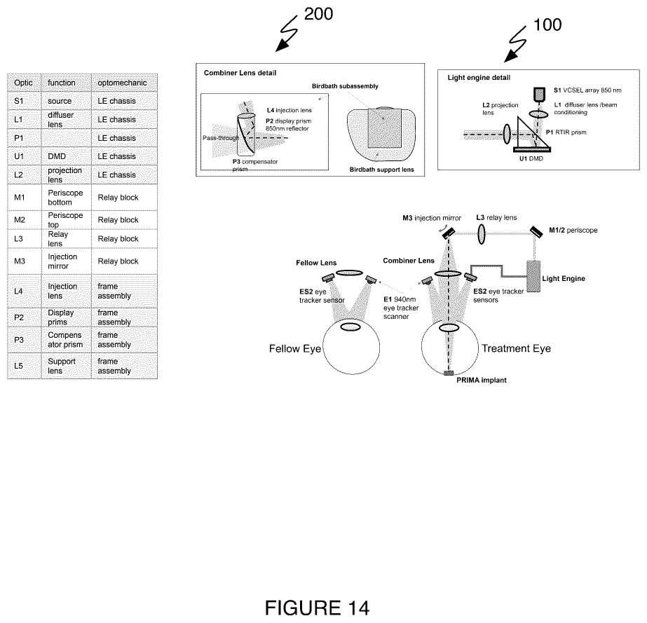

The light engine 100 functions to emit patterned light. The light engine 100 can transmit light to the combiner lens 200, transmit light to one or more other optical components (e.g., a lens, a mirror, a filter, etc.), transmit light to the target, and/or otherwise interface with the target and/or one or more system components. In a specific example, the light source 100 can transmit light to the combiner lens 200 directly or via one or more optical components. Examples are shown in FIG. 8 and FIG. 14. The light engine 100 can optionally be or be part of a projector subsystem. For example, the system can include a projector that includes the light engine 100. In a specific example, the system can include a projector that includes the light engine 100 and the combiner lens 200.

In an example, the patterned light can be a set of patterned light frames (e.g., a time series of patterned light frames). The patterned light can optionally be defined by a set of light pattern parameters. In an example, the light pattern parameters can prescribe a time series of patterned light frames, wherein one or more patterned light frames encode a frame of content. Each patterned light frame can optionally be produced by an array (e.g., two-dimensional array) of optical components (e.g., light sources, mirrors, display elements, etc.). The light pattern parameters can be determined based on measurements from the external sensor 300, measurements from the eye sensor 400, a target location (e.g., wherein the target location is determined based on measurements from the eye sensor 400), a target orientation (e.g., wherein the target orientation is determined based on measurements from the eye sensor 400), a location of the projected patterned light (e.g., the most recent projected patterned light frame) on the target, an orientation of the projected patterned light (e.g., the most recent projected patterned light frame) on the target, content (e.g., a frame of content), current or previous light pattern parameters, current or previous steering module parameters, calibration information, a mapping of cells (e.g., retinal ganglion cells) in the target, and/or any other suitable information. The light pattern parameters can be determined using the processing system 500, using a model, manually, randomly, predetermined, and/or otherwise determined.

Light pattern parameters can include global parameters defining the entire array of display elements and/or local parameters defining one or more individual display elements. In a specific example, a set of light pattern parameters can define each patterned light frame. Examples of global parameters and/or local parameters can include: origin location (e.g., location of the origin of the patterned light, location of a display element, etc.), patterned light direction (e.g., direction of the optical path of the projected patterned light, direction of the optical path of projected light from a display element, etc.), amplitude, phase, polarization, wavelength, power (e.g., brightness), spatial parameters (e.g., across the array of display elements), temporal parameters (e.g., a sequence of light pattern parameters defining a set of patterned light frames; a time associated with a display element being on or off; etc.), and/or any other light parameters. Light pattern parameters can be fixed (e.g., a fixed wavelength, fixed intensity, etc.) and/or variable.

The light engine 100 can optionally include one or more optical components. Examples of optical components include: light sources, mirrors, lenses (e.g., microlens, diffractive lens, metalens, etc.), light modulators, diffractive components, diffusers, filters, fibers, waveguides, prisms, dichroics (e.g., dichroic mirrors), irises, apertures, periscopes, back reflectors, light modulators, and/or any other optical components. Optical components can optionally include adaptive (e.g., actuated) optical components.

The light engine 100 can include a set of light sources 120 (e.g., an array of light sources). Examples of light sources include LEDs (e.g., μLEDs), lasers (e.g., vertical-cavity surface-emitting laser (VCSEL), fiber coupled lasers, laser diodes, etc.), lamps (e.g., incandescent lamps), and/or any other light source. In a specific example, the set of light sources 120 (e.g., array of light sources) can be or include a set of vertical-cavity surface-emitting lasers (e.g., an array of vertical-cavity surface-emitting lasers). The light emitted by a light source can include visible light, ultraviolet light, infrared light (e.g., near-infrared light), and/or any other light wavelength. The number of light sources in the set of light sources 120 can be 2-10million or any range or value therebetween (e.g., at least 2, at least 4, at least 8, at least 10, at least 20, at least 50, at least 100, at least 1000, etc.). In an example, the set of light sources 120 can form an array (e.g., a two-dimensional grid). The shape of the array can be a square, a rectangle, a pentagon, a hexagon, an octagon, a circle, and/or any other shape. For example, a square or rectangular array can be 1×2 (e.g., 2 light sources)−10,000×10,000 or any range or value therebetween (e.g., at least 2×2, at least 4×4, at least 10×20, 16×26, etc.), but can alternatively be larger than 10,000×10,000.

The light engine 100 can optionally include one or more diffusers in front of the set of light sources 120. In an example, a diffuser (e.g., an engineered diffuser) can adjust the cone angle and/or cone direction of light leaving the diffuser to match the acceptance cone of one or more optical components (e.g., lens, prism, the display module 140, etc.).

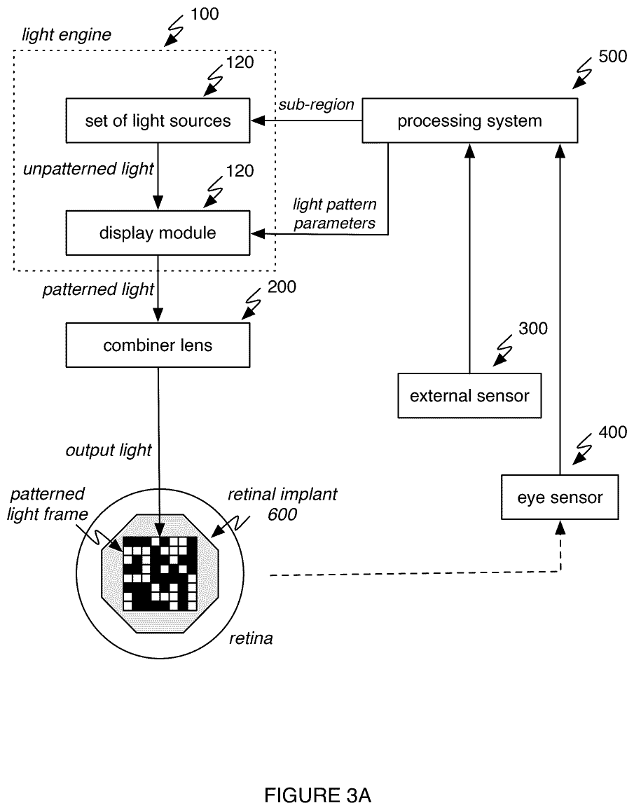

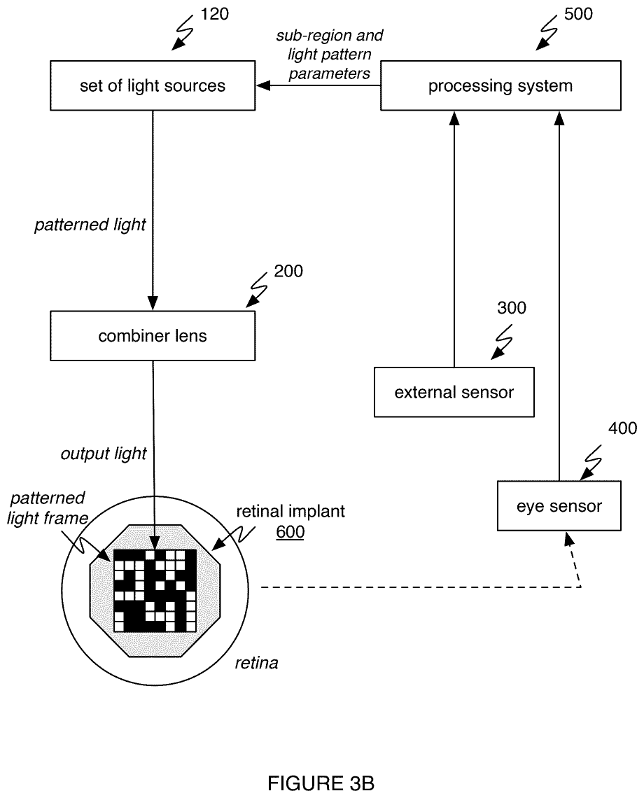

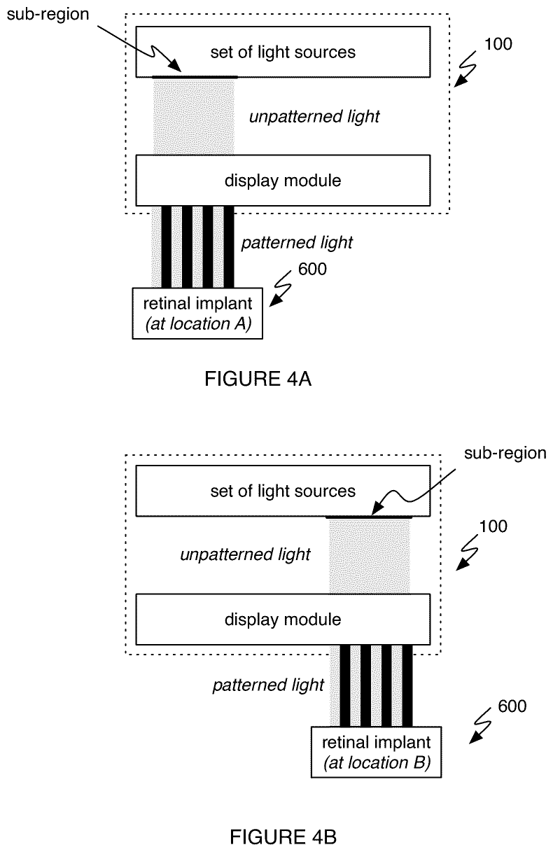

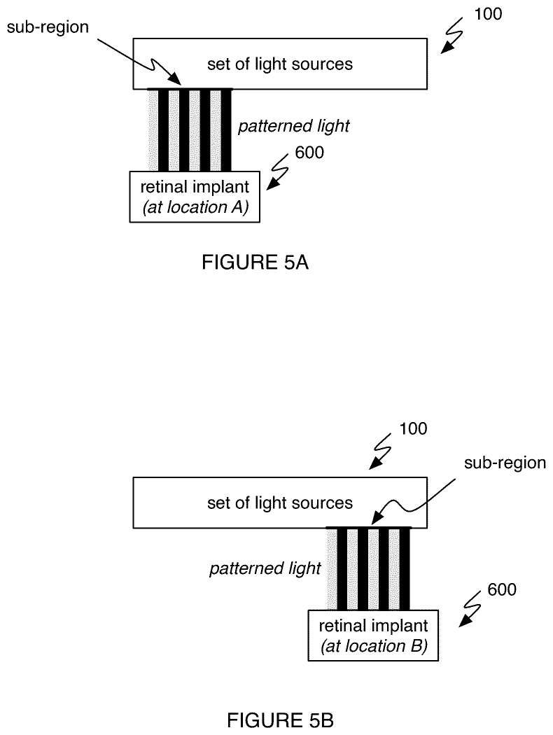

The light engine 100 can optionally include a display module 140. In a first variant, the light engine 100 can include a set of light sources 120 and a separate display module 140. In an example, the display module 140 can be configured to modulate unpatterned (e.g., uniform) light from the set of light sources 120 to output patterned light. Examples are shown in FIG. 3A, FIG. 4A, and FIG. 4B In a specific example, the light engine 100 can include a set of light sources 120 (e.g., a VCSEL array), a diffuser (e.g., an engineered diffuser), one or more lenses, a prism, and a display module 140 (e.g., spatial light modulator). An example is shown in FIG. 9. In a second variant, the set of light sources 120 can function as a display (e.g., where each light source is a pixel in the display). Examples are shown in FIG. 3B, FIG. 5A, and FIG. 5B. For example, the set of light sources 120 can be a high-density array of light sources (e.g., a high density μLED array).

In an example, the display module 140 can include a spatial light modulator (SLM). In specific examples, the SLM can include a MEMS-based SLM (e.g., a digital micromirror device (DMD)), a LC-based SLM, and/or any other type of SLM. The SLM can be an amplitude modulator and/or a phase modulator. The SLM preferably includes a binary SLM, but can additionally or alternatively include a non-binary SLM. The display module 140 can operate between 100 FPS-100,000 FPS or any range or value therebetween (e.g., 10,000 FPS-50,000 FPS; 32,000 FPS; at least 10,000 FPS; at least 20,000 FPS; at least 30,000 FPS; etc.). The display module 140 can optionally include an array of display elements which function to produce the patterned light (e.g., based on the light pattern parameters). Each display element can include one or more optical components, actuators, and/or any other suitable elements. In a specific example, each display element can be or include a mirror. In another specific example, the display module 140 can include one or more actuators, wherein each actuator drives a display element. The array of display elements is preferably two-dimensional, but can alternatively be one-dimensional, three-dimensional, and/or any other number of dimensions. Each display element can be binary (e.g., on versus off; on-target versus deflected off-target; etc.), discrete (e.g., high intensity, low intensity, off), continuous, and/or otherwise configured.

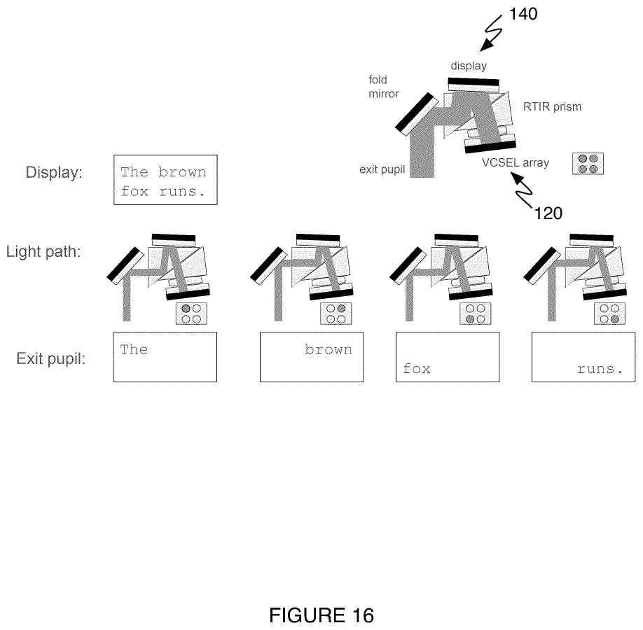

In variants, the light engine can include a set of light sources 120 (e.g., array of light sources), wherein all or a portion of one or more sub-regions of the set of light sources 120 can be selectively illuminated (e.g., as controlled by the processing system 500). In an example, selectively illuminating a sub-region of the set of light sources 120 can include illuminating all or a portion of light sources within the sub-region of the set of light sources 120 while light sources outside the sub-region of the set of light sources 120 are not illuminated. Examples are shown in FIG. 4A, FIG. 4B, FIG. 5A, FIG. 5B, and FIG. 16. As used herein, a sub-region of the set of light sources 120 refers to a subset of the set of light sources 120. The sub-region of the set of light sources 120 is preferably a contiguous region, but can alternatively be noncontiguous (e.g., including a first subset of light sources and a second subset of light sources, where the second subset of light sources is separated from the first subset of light sources). In an example, all or a portion of the light sources within the sub-region are illuminated while no light sources outside the sub-region are illuminated. In a first example, all light sources within the selected sub-region of the set of light sources 120 are illuminated uniformly. In a second example, light sources within the selected sub-region of the set of light sources 120 are individually controlled according to the light pattern parameters (e.g., such that a portion of the light sources within the selected sub-region may not be illuminated).

In a first variant, the set of light sources 120 functions as a display, wherein the selected sub-region of the set of light sources 120 includes light sources functioning as pixels (e.g., light sources within the sub-region are individually controlled according to the light pattern parameters). In a second variant, the display module 140 is modulated in tandem with the selective illumination of the set of light sources 120, such that the portion of the display module 140 receiving light from the selected sub-region is controlled to pattern the light according to the light pattern parameters. In a third variant, the entire display module 140 is patterned according to the light pattern parameters, such that any subset of the display module 140 receiving light from the selected sub-region is patterned. In a fourth variant, the display module 140 includes multiple sub-display modules, each sub-display module corresponding to a sub-region of the set of light sources 120 that can be selected for illumination. In an example, each sub-display module is configured to pattern light according to the light pattern parameters, such that any selected sub-region of the set of light sources 120 will project light onto a corresponding sub-display module to pattern the light accordingly.

The light engine 100 can be coupled to the combiner lens 200, the glasses frame, and/or any other system components. In a first example, the light engine 100 can be coupled to the rim of the glasses and/or the lens of the glasses. In a second example, the light engine 100 can be coupled to the temple of the glasses.

However, the light engine 100 can be otherwise configured.

### 3.2. The Combiner Lens 200

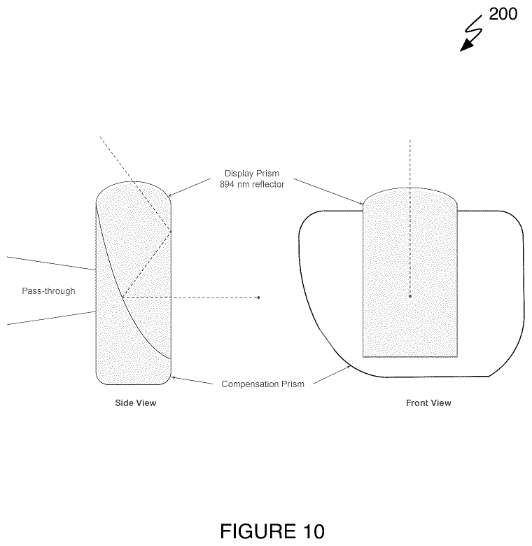

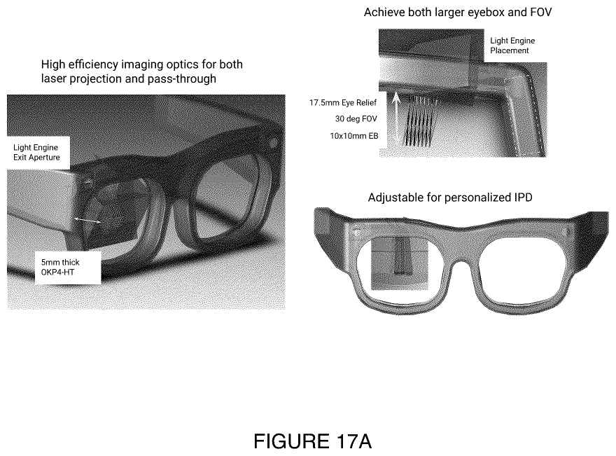

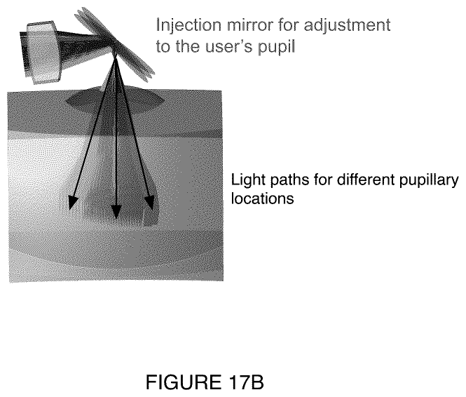

The system can optionally include a combiner lens 200, which functions to direct the patterned light from the light engine 100 towards the target (e.g., the retinal implant 600). Examples are shown in FIG. 10, FIG. 17A, and FIG. 17B.

In an example, the combiner lens 200 can direct the patterned light onto the target within a threshold distance of the target location and/or direct the patterned light such that it is centered on the target location within a threshold center offset. The threshold distance can optionally be between 0-5 mm or any range or value therebetween (e.g., within 200 nm, within 500 nm, within 1000 nm, within 5000 nm, within 0.01 mm, within 0.05 mm, within 0.1 mm, within 0.5 mm, within 1 mm, within 2 mm, etc.). The threshold center offset can optionally be between 0-1 mm or any range or value therebetween (e.g., within 200 nm, within 500 nm, within 1000 nm, within 5000 nm, within 0.01 mm, within 0.05 mm, within 0.1 mm, within 0.5 mm, within 1 mm, etc.).

The combiner lens 200 can include one or more optical components. In a specific example, the combiner lens 200 can include a display prism and/or a compensator prism. In a specific example, the combiner lens 200 can optionally include an injection lens (e.g., to compensate for different injection angles). The patterned light from the light engine 100 can optionally be directed to the combiner lens 200 via one or more optical components (e.g., a periscope, a relay lens, and/or an injection mirror).

The combiner lens 200 can be coupled to the light engine 100, the glasses frame, and/or any other system components. In a first example, the combiner lens 200 can be positioned in front of a lens of the glasses (e.g., in front of the right or left glasses lens). In a second example, the combiner lens 200 can be positioned behind a lens of the glasses (e.g., behind the right or left glasses lens). The combiner lens 200 is preferably partially or completely free of occlusions (e.g., to allow for the user to maintain all or a portion of their peripheral vision), but can alternatively not be free of occlusions.

However, the combiner lens 200 can be otherwise configured.

The position of the combiner lens 200 and/or the light engine 100 can optionally be adjustable. For example, the position of the combiner lens 200 and/or the light engine 100 within or on the glasses frame can optionally be adjustable. In a specific example, the position of the combiner lens 200 and/or the light engine 100 on the glasses frame can be determined based on an inter-pupillary distance of the user (e.g., a measured inter-pupillary distance of the user). In an example, the combiner lens 200 and/or the light engine 100 can be linearly actuated (e.g., along a rail system) to adjust the target location for the patterned light. The combiner lens 200 and/or the light engine 100 can optionally be adjusted at least a threshold distance (e.g., at least 1 mm, at least 2 mm, at least 3 mm, at least 4 mm, at least 5 mm). The positions that the combiner lens 200 and/or the light engine 100 can occupy can be discrete (e.g., a set of defined positions) or continuous. In a specific example, this adjustment can accommodate different interpupillary distances, enabling the system to project patterned light through the pupil with increased accuracy and across a range of users with different anatomies. In a specific example, the user can undergo a calibration phase wherein the position of the combiner lens 200 and/or the light engine 100 is adjusted (e.g., manually or automatically) based on the user's interpupillary distance (e.g., a measured interpupillary distance).

### 3.3. The External Sensor 300

The system can optionally include an external sensor 300, which functions to acquire content (e.g., images, videos, and/or other measurements), wherein the content can be used to determine the light pattern parameters. For example, the external sensor 300 can function to collect an image, wherein the light pattern parameters are determined such that all or a portion of the image is projected onto the target (e.g., as described below). The external sensor 300 can transmit measurements to the processing system 500 and/or otherwise interface with one or more system components.

The number of external sensors can optionally be between 1-5 or any range or value therebetween (e.g., 1, 2, 3, 4, etc.), but can alternatively be greater than 5. In a first specific example, the system includes a single external sensor. In a second specific example, the system includes two external sensors.

The external sensor 300 is preferably a camera, but can additionally or alternatively include light sensor (e.g., camera, photodiode, etc.), motion sensor (e.g., inertial measurement unit, retroreflector, other optical components, etc.), depth sensor, a depth sensor (e.g., range finding sensor), thermal sensor, electrodes, and/or any other sensor. In an example, the external sensor 300 can be mounted on the glasses such that images acquired by the external sensor 300 partially or fully overlap with the field of view of the user's eyes.

However, the external sensor 300 can be otherwise configured.

The system can optionally include one or more supplemental sensors. Examples of supplemental sensors include: a laser safety sensor, a depth sensor (e.g., range finding sensor; to receive depth information about an object in the field of view of the external sensor), and/or any other supplemental sensors.

### 3.4. The Eye Sensor 400

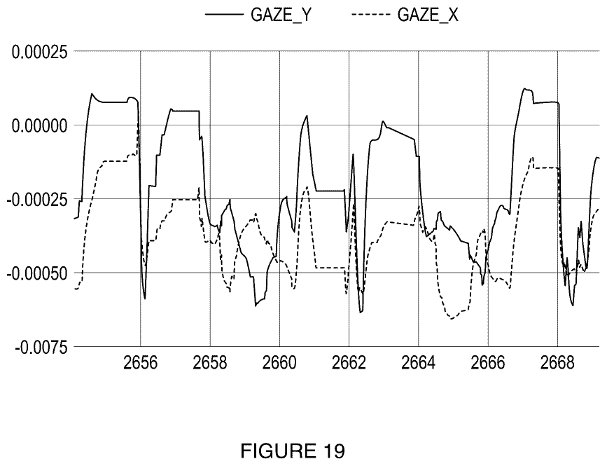

The eye sensor 400 functions to: image and/or track all or a portion of the target (e.g., the retinal implant), image and/or track all or a portion of the eye (e.g., the pupil), image and/or track the patterned light frame on the target, provide feedback for one or more system components (e.g., the light engine 100, the retinal implant 600, etc.), and/or collect any other measurements. An example is shown in FIG. 19. Additionally or alternatively, the eye sensor 400 can function to acquire measurements for diagnostics. The eye sensor 400 can transmit measurements to the processing system 500 and/or otherwise interface with one or more system components.

In an example, the eye sensor 400 can be used to sample measurements of one or more eye features (e.g., to image and/or track the one or more eye features). Examples of eye features include: the pupil (e.g., the center of the pupil), corneal reflection, the retinal implant 600, a retinal feature, a feature of the retinal implant 600 (e.g., visual feature of the retinal implant), a portion thereof, and/or any other eye features. Specific examples of retinal features include: one or more individual retinal cells, vasculature, visual streak, optic disk, fluorescent marker, a feature of the retinal implant 600, a visual feature of projected light on the retina and/or the retinal implant 600, and/or any other visual features of the retina and/or the retinal implant 600. Specific examples of features of the retinal implant 600 include the center of the retinal implant 600, pixels of the retinal implant 600, project light reflecting off the retinal implant 600, light projected from a light source in the retinal implant 600, and/or any other visual features of the retinal implant 600. In an example, light from the light engine 100 can reflect off the eye feature(s) to the eye sensor 400 (e.g., directly or via one or more optical components). In a specific example, tracking the retinal implant 600 (e.g., rather than the pupil) can account for different placements of the retinal implant 600 and/or anatomical differences, which can provide more accurate light projection onto the retinal implant 600.

The eye sensor 400 can include one or more sensors (e.g., at least two sensors, at least three sensors, etc.). In a first example, the eye sensor 400 can include a first sensor configured to collect a set of measurements (e.g., images) of a left eye and a second sensor configured to collect a set of measurements (e.g., images) of a right eye. In a second example, the eye sensor 400 can include a single sensor configured to collect a set of measurements (e.g., images) of one eye (e.g., the left or the right eye). Examples of sensors in the eye sensor 400 can include: a light sensor (e.g., camera, photodiode, etc.), motion sensor (e.g., inertial measurement unit, retroreflector, other optical components, etc.), and/or any other sensor. The light sensor can measure infrared light, visible light (e.g., RGB), UV light, and/or any other wavelength. In an example, the eye sensor 400 can be configured to sample a set of measurements of the eye (e.g., of an eye feature, a retinal feature, a retinal implant feature, etc.) using infrared light. In a first specific example, the eye sensor 400 can include one or more cameras (e.g., full-field camera, scanning cameras, etc.). In a second specific example, the eye sensor 400 can include a micro-electromechanical (MEMS) eye tracker (e.g., including a MEMS micromirror and photodetectors). The eye sensor 400 can optionally include one or more light sources (e.g., LEDs, laser, any light source as described above, etc.). The eye sensor 400 can optionally include one or more optical components.

However, the eye sensor 400 can be otherwise configured.

### 3.5. The Processing System 500

The processing system 500 functions to control the light engine 100 and/or otherwise control one or more system components. The processing system 500 can receive measurements (e.g., images, eye tracking information, etc.) from the external sensor 300 and/or the eye sensor 400.

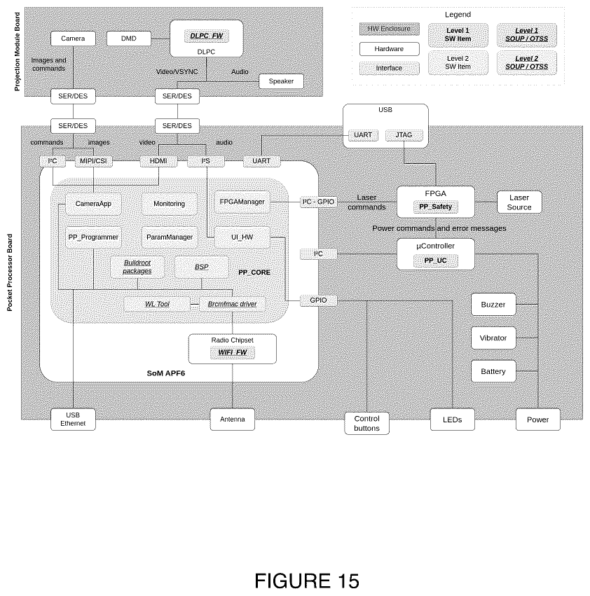

The processing system 500 can include one or more: CPUs, GPUs, TPUs, custom FPGA/ASICS, microprocessors, servers, cloud computing, and/or any other suitable components. An example is shown in FIG. 15. The processing system 500 can be local (e.g., providing onboard compute), remote (e.g., cloud computing server, etc.), distributed, and/or otherwise arranged relative to any other system or module.

The processing system 500 can function to perform all or parts of the method. In a first variant, the processing system 500 can determine the target location. For example, the processing system 500 can detect and/or track a location of one or more eye features (e.g., a target feature, a tracked gaze, etc.) based on measurements received from the eye sensor 400, and determine the target location based on the tracked location of the one or more eye features. In a second variant, the processing system 500 can determine the sub-region of the set of light sources 120 based on the target location. For example, the processing system 500 can determine the sub-region of the set of light sources 120 to selectively illuminate based on the target location. In a third variant, the processing system 500 can determine content. For example, the processing system 500 can determine the content (e.g., an image) based on measurements received from the external sensor 300. In a fourth variant, the processing system 500 can control the light engine 100 based on the sub-region and/or the content. In a first example, the processing system 500 can control the light engine 100 (e.g., the set of light sources 120 within the light engine 100) based on the sub-region. In a second example, the processing system 500 can determine light pattern parameters based on the content, and control the light engine 100 (e.g., the display module 140 within the light engine 100 and/or the set of light sources 120 within the light engine 100) based on the light pattern parameters. In a fifth variant, the processing system 500 can perform two or more of the previous variants.

The processing system 500 can optionally include or use one or more models, including one or more rendering models, light propagation models, tracking models, and/or any other model. The models can include classical or traditional approaches, machine learning approaches, and/or be otherwise configured. The models can include regression, decision tree, clustering, association rules, dimensionality reduction, neural networks (e.g., GNN, CNN, DNN, CAN, LSTM, RNN, FNN, encoders, decoders, deep learning models, transformers, etc.), ensemble methods, optimization methods, classification, rules, heuristics, equations (e.g., weighted equations, etc.), selection, lookups, regularization methods (e.g., ridge regression), Bayesian methods (e.g., Naive Bayes, Markov, etc.), instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministics, genetic programs, foundation models (e.g., language models), computer vision models (e.g., feature extractors, segmentation models, object detectors, etc.), and/or any other suitable model.

Models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated.

However, the processing system 500 can be otherwise configured.

### 3.6. Retinal Implant 600

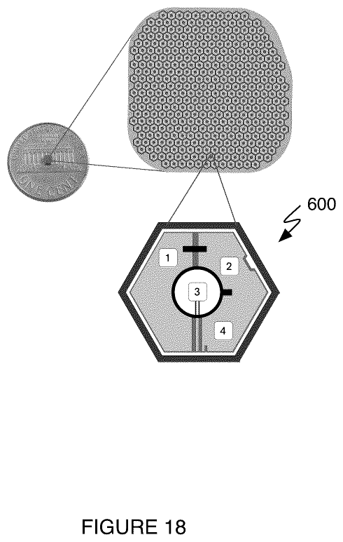

The system can optionally include a retinal implant 600, which functions to emit excitation signals that are received by retinal cells (e.g., genetically modified cells and/or non-modified cells), in response to receiving light projected by the light engine 100 (e.g., through the combiner lens 200). The excitation signals (e.g., electrical signals) can encode content (e.g., visual data, sensory data, other external information, artificial data, any other data, etc.). An example is shown in FIG. 18. For example, the light engine 100 and/or the combiner lens 200 can project patterned light onto the retinal implant 600, wherein the retinal implant 600 is configured to generate electrical signals in response to receiving the patterned light.

The retinal implant 600 can be implanted on or below the retina. In specific examples, the retinal implant 600 can be a subretinal implant or an epiretinal implant. In specific examples, the retinal implant can include systems as described in U.S. application Ser. No. 16/631,029 filed 14 Jan. 2020, U.S. application Ser. No. 17/296,685 filed 25 May 2021, U.S. application Ser. No. 17/593,274 filed 14 Sep. 2021, U.S. application Ser. No. 19/188,842 filed 24 Apr. 2025, and/or U.S. application Ser. No. 18/740,854 filed 12 Jun. 2024, and/or U.S. application Ser. No. 19/210,450 filed 16 May 2025, each of which is incorporated in its entirety by this reference.

The retinal implant 600 can include one or more pixels, wherein each pixel includes circuitry and/or components configured to receive a light signal (e.g., patterned light) and generate excitation signals (e.g., electrical signals) in response to receiving the light signal. In specific examples, pixels can include photovoltaic cells, photodiodes, any light sensor system, any electrical emission system (e.g., electrodes), any light emission system (e.g., μLEDs, etc.), and/or any other optical components. In a specific example, the retinal implant 600 can operate at or near cellular resolution (e.g., one pixel can excite less than 20 cells, less than 10 cells, less than 5 cells, less than 2 cells, 1 cell, etc.). The number of pixels can be between 100-50,000 or any range or value therebetween (e.g., at least 1000; at least 2000; at least 5000; at least 10000; etc.), but can alternatively be less than 100 or greater than 50,000.

The retinal implant 600 can optionally include and/or be coupled (e.g., adhered, mounted, etc.) to one or more optical components (e.g., microoptical components). The optical component(s) can function to collimate light, homogenize light, focus light (e.g., onto the light sensor systems), reduce light shining back through the eye lens, and/or otherwise modify light emitting to or from the display 100. The optical component(s) can be located on the front of the retinal implant 600 (facing the retina) and/or back of the retinal implant 600.

However, the retinal implant 600 can be otherwise configured.

### 3.7. User Interface Components

The system can optionally include one or more user interface components, which can function to receive information from the user and/or communicate information to the user. User interface components can include output components and/or input components. Output components can function to communicate information to the user. Examples of information provided to a user via one or more output components include: a confirmation of a user selection and/or user input, depth information, power information, system state information, and/or any other information. Examples of output components can include: speakers, a visual display (e.g., on a lens of the glasses), haptic feedback system, and/or any other components providing information to the user. In a specific example, one or more speakers can optionally be used for noise canceling. Input components can function to receive one or more user inputs. Examples of user inputs received from a user via one or more input components include: a mode selection (e.g., reading mode, zoom mode, target mode, etc.), a volume selection (e.g., raise volume, lower volume, target volume level, etc.), a zoom selection (e.g., increase zoom, decrease zoom, target zoom level, etc.), a brightness selection (e.g., increase brightness, decrease brightness, target brightness level, etc.), a contrast selection (e.g., increase contrast, decrease contrast, target contrast, etc.), power, and/or any other information. Examples of input components can include: a microphone (e.g., to receive voice commands), buttons (e.g., to receive user inputs), and/or any other components receiving information to the user. In a specific example, voice commands can include a zoom adjustment command to adjust the camera zoom.

However, the one or more user interface components can be otherwise configured.

However, the system can be otherwise configured.

### 4. Method

As shown in FIG. 2, the method can include: determining a target location S100, determining a sub-region of a set of light sources based on the target location S200, determining content S300, and producing patterned light based on the sub-region and the content S400. The method can optionally include receiving one or more user inputs via one or more user interface components (e.g., via one or more input components). The method can optionally include providing one or more output via one or more user interface components (e.g., via one or more output components). However, the method can include any other suitable steps.

The method can be performed by one or more components of the system. All or portions of the method can be performed automatically, manually, semi-automatically, and/or otherwise performed. All or portions of the method can be performed in real time, iteratively, concurrently, asynchronously, periodically, and/or at any other suitable time.

Determining a target location S100 functions to determine the location for projecting the patterned light. The target location can be the location of the target (e.g., the center of the target), a region encompassing the target (e.g., a region of the retina that includes the target), a location and/or region within the target (e.g., a target location on the retina, a target location on the retinal implant 600, etc.), an eye feature, and/or any other location associated with the target and/or the eye. In a specific example, the target location can be the location of the retinal implant 600 (e.g., of the center of the retinal implant 600). The target location can optionally be determined by the processing system 500, the eye sensor 400, a combination thereof, and/or any other system components.

Determining a target location S100 can optionally include sampling a set of measurements of an eye feature, and determining the target location (e.g., tracking a target location) based on the measurements of an eye feature. In a first specific example, the target location can be the location of an eye feature. In a second specific example, the target location can be determined based on the location of an eye feature (e.g., a predetermined distance and/or transformation relative to the eye feature). The set of measurements of the eye feature can optionally be collected by the eye sensor 400. For example, determining the target location can include detecting and/or tracking a location of one or more eye features based on the set of measurements collected via the eye sensor 400 (e.g., tracking the user's gaze, tracking the retinal implant 600, etc.). In specific examples, the set of measurements of the eye feature can be measurements of the exterior of the eye, the pupil of the eye, the retina of the eye, the retinal implant 600, and/or any other eye feature.

In variants, a tracking model can output tracked coordinates of the eye feature, a tracked gaze, and/or any other suitable outputs. Inputs to the tracking model can include measurements (e.g., images) received from the eye sensor 400. In examples, the tracking model can use a deep-learning based approach (e.g., using an NN to segment an image frame, and post-processing the segmented image to determine a pupil contour), a computer-vision approach (e.g., pre-processing an image frame to generate a binary image, and using blob detection to determine a pupil contour), and/or any other image analysis methods. Outputs from the tracked model can optionally be processed. Examples of processing include filtering, affine transformation, scaling, and/or any other processing methods. In an example, the tracking model can output coordinates of the eye feature.

However, the target location can be otherwise determined.

Determining a sub-region of a set of light sources based on the target location S200 functions to select a subset of the set of light sources 120 for illumination.

The sub-region of the set of light sources 120 can be determined based on the target location. For example, the sub-region can be determined (e.g., selected) such that the patterned light projects onto the retinal implant (e.g., with minimal or no projection of light beyond the edges of the retinal implant). In a specific example, the sub-region can be selected such that light originating from the light sources within the sub-region of the set of light sources 120 are projected onto the target within a threshold distance of the target location and/or centered on the target location within a threshold center offset. The threshold distance can optionally be between 0-5 mm or any range or value therebetween (e.g., within 200 nm, within 500 nm, within 1000 nm, within 5000 nm, within 0.01 mm, within 0.05 mm, within 0.1 mm, within 0.5 mm, within 1 mm, within 2 mm, etc.). The threshold center offset can optionally be between 0-1 mm or any range or value therebetween (e.g., within 200 nm, within 500 nm, within 1000 nm, within 5000 nm, within 0.01 mm, within 0.05 mm, within 0.1 mm, within 0.5 mm, within 1 mm, etc.).

In an example, a sub-region of the set of light sources 120 that is illuminated can correspond to a sub-field of view (FOV) within a full FOV. The full FOV (e.g., the FOV that is accessible via the entire set of light sources 120) is preferably at least 20 degrees (e.g., at least 25 degrees, at least 30 degrees, etc.), but can alternatively be less than 20 degrees. The effective eye box size (e.g., corresponding to the full FOV) is preferably at least 2 mm (e.g., at least 5 mm, at least 6 mm, at least 10 mm, etc.), but can alternatively be less than 2 mm. In variants, this selective illumination (e.g., dynamic illumination) can provide a larger accessible eye box while limiting the amount of projected light illuminating portions of the eye beyond the retinal implant. In a specific example, as the target location is updated (e.g., as the eye feature is tracked), the sub-region can be dynamically updated (e.g., iteratively as new measurements are acquired) to continually re-align the sub-FOV with the retinal implant.

The sub-region can optionally be determined by identifying a target light source in the set of light sources 120 that corresponds to the target location (e.g., the light source projecting light closest to the target location is selected as the sub-region), and determining the sub-region based on the target light source. In a first variant, the sub-region can be a sub-region of the set of light sources 120 that encompasses a predetermined number of light sources surrounding the target light source. In a second variant, the sub-region can be a sub-region of the set of light sources 120 that encompasses light sources within a predetermined distance from the target light source. In a third variant, the sub-region can be selected from a predetermined set of sub-regions. For example, light sources in the set of light sources 120 can be grouped into predetermined sub-regions (e.g., each including a subset of the set of light sources 120), wherein, the sub-region that includes the target light source is selected. The sub-regions can include overlapping subsets of light sources (e.g., a single light source can be within two sub-regions) or nonoverlapping subsets of light sources (e.g., each light source is within a single sub-region).

However, the sub-region can be otherwise selected.

Determining content S300 functions to determine information to encode in the projected light. The content is preferably visual content, but can alternatively be any other content (e.g., content for other senses, such as temperature, other information not related to senses, etc.). Examples of content include: measurements (e.g., images, videos, etc.) of a real-world scene; artificial reality (AR) overlays; AR overlaid onto measurements of a real-world scene; virtual reality (VR); text; and/or any other information. In an example, the content can be measurements captured by the external sensor 300. In a specific example, the content can be one or more images sampled by the external sensor 300 (e.g., a frame of a video).

Determining the content can optionally include processing the content. For example, determining the content can include processing the measurements (e.g., a set of images) received from the external sensor 300. Processing images can include: cropping, downsampling, filtering, thresholding (e.g., binary thresholding, grayscale threshold, etc.), edge detection, contrast enhancement, and/or any other image processing techniques. The content can optionally be processed using a model (e.g., an artificial intelligence engine). In a first example, an image can be cropped based on measurements from the eye sensor 400 (e.g., based on the tracked eye feature). In a specific example, the image can be cropped to correspond to the portion of the image that the user is viewing, based on their eye orientation. In a second example, the image can be cropped based on a user input (e.g., a zoom selection).

The content can optionally be processed based on a mode. In a first variant, in a reading mode, the content can be processed to enhance text and/or zoom in on text. For example, a text recognition model can be used to identify text in an image, and a processing model can be used to adjust display of the image based on the identified text. In an illustrative example, the font of the text can be adjusted (e.g., from a serif font to a sans serif font). In a second variant, in a zoom mode, processing the content can include cropping an image received from the external sensor 300 based on a zoom selection. In a specific example, the zoom selection can be received from the user (e.g., via an input component of the user interface). In a third variant, in image recognition mode, processing the content can include using an image recognition model to identify an object in an image, and using an image processing model to adjust display of the image based on the identified object.

However, content can be otherwise determined.

Producing patterned light based on the sub-region and the content S400 functions to project the patterned light onto the target. In an example, S400 can include: controlling the light engine 100 based on the sub-region, determining light pattern parameters based on content, and controlling the light engine 100 based on the light pattern parameters.

Producing patterned light can include controlling the light engine 100 based on the sub-region of the set of light sources 120. In variants, all or a portion of the set of light sources 120 within the sub-region can be illuminated. For example, all or a portion of the light sources within the sub-region can be illuminated while no light sources outside the sub-region are illuminated. In an example, the method can include: selecting a sub-region of the set of light sources 120 (e.g., array of light sources) based on a target location (e.g., tracked location of a retinal feature), and producing patterned light (e.g., encoding the content) by selectively illuminating at least a portion of the sub-region of the set of light sources 120, wherein each light source in the set of light sources 120 located outside the sub-region of the array of light sources is not illuminated. In a first example, all light sources within the selected sub-region of the set of light sources 120 are illuminated uniformly. In a second example, light sources within the selected sub-region of the set of light sources 120 are individually controlled (e.g., such that a portion of the light sources within the selected sub-region may not be illuminated). In a specific example, producing the patterned light includes: determining a subset of the sub-region of the set of light sources 120 based on the content, and illuminating the subset of the sub-region of the set of light sources 120 to produce the patterned light.

The sub-region of the set of light sources 120 can optionally be controlled based on a user input. In a specific example, the power of the sub-region of the set of light sources 120 can be controlled on a brightness selection.

Producing patterned light can include controlling the light engine 100 based on the content. In an example, the patterned light can encode the content. For example, the light engine 100 can be controlled based on light pattern parameters, wherein the light pattern parameters are determined based on the content. In a specific example, when the content is a set of images, the patterned light can be a time series of patterned light frames, wherein each patterned light frame corresponds to an image (e.g., a frame) of the set of images. In an example, a rendering model can output light pattern parameters based on content. In a specific example, the rendering model can output light pattern parameters for one or more patterned light frames based on a content frame.

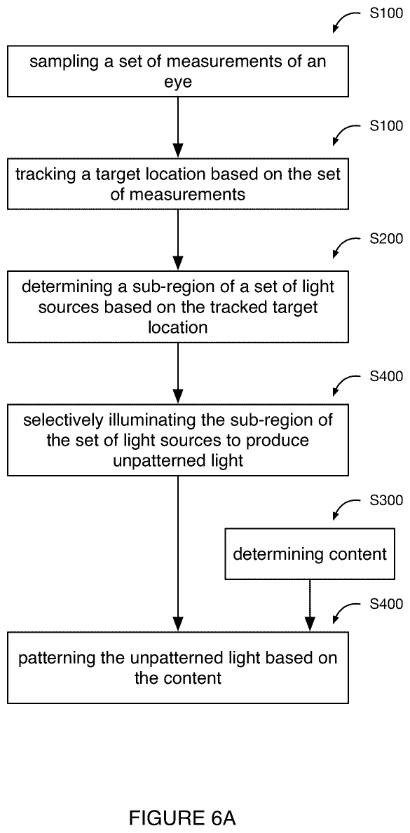

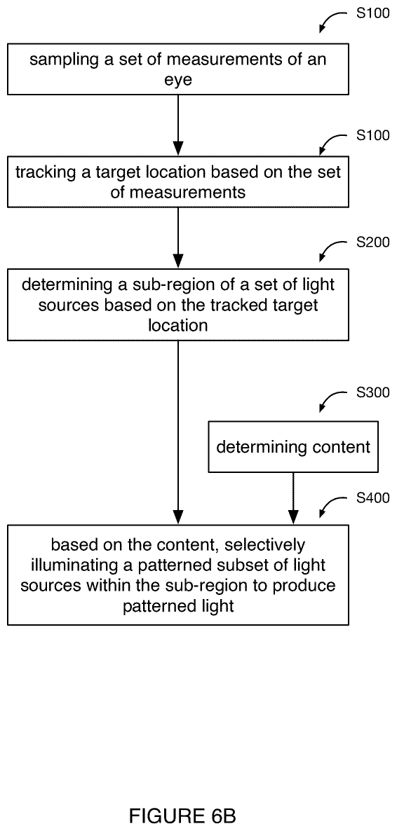

In a first variant, the display module 140 can be controlled based on the content (e.g., an image). An example is shown in FIG. 6A. In an example, one or more display elements (e.g., mirrors) of the display module 140 can be controlled (e.g., oriented) based on the content. In a specific example, when the content includes an image, each display element (e.g., mirror) of the display module 140 can represent a pixel of the image. In an example, the sub-region of set array of light sources 120 projects unpatterned light onto a corresponding sub-region of the display module 140 (e.g., a spatial light modulator), wherein the corresponding sub-region of the the display module 140 modulates the unpatterned light to output the patterned light. In a specific example, when the display module 140 includes a digital micromirror device, the sub-region of the display module 140 (corresponding to the sub-region of the set of light sources 120) can include a subset of mirrors of the digital micromirror device. In a specific example, each display element within the sub-region of the display module 140 can correspond to a pixel of a cropped image (e.g., a portion of the image that the user is viewing).

In a first embodiment, the entire display module 140 is patterned based on the content. In an example, the display module 140 is patterned according to the content (e.g., based on the light pattern parameters), wherein a subset of the display module 140 is illuminated by the sub-region of the set of light sources 120. In a specific example, the display module 140 can include a digital micromirror device, wherein patterning the display module 140 based on the content can include determining an orientation of each mirror based on the content. In an example, the display module 140 can be patterned based on an image, wherein the subset of the display module 140 illuminated by the sub-region of the set of light sources 120 corresponds to a cropped portion of the image. In a second embodiment, a portion of the display module 140 is patterned based on the content. In an example, a subset of the display module 140 is illuminated by the sub-region of the set of light sources 120, wherein this subset of the display module 140 is patterned based on the content. In a specific example, the display module 140 can include a digital micromirror device, wherein controlling the display module 140 based on the content can include determining, based on the content, an orientation of each mirror of the digital micromirror device within the subset of the digital micromirror device illuminated by the sub-region of the set of light sources 120. In an example, the subset of the display module 140 can be patterned based on a cropped portion of an image.

In a second variant, the set of light sources 120 can be controlled based on the content. An example is shown in FIG. 6B. For example, the set of light sources 120 can form a display, wherein the light sources within the sub-region can be controlled according to the light pattern parameters. In a specific example, when the content includes an image, each light source within the set of light sources 120 can correspond to a pixel of the image. In an example, a subset of the sub-region of the set of light sources 120 can be selectively illuminated according to the light pattern parameters (e.g., such that light sources in the sub-region of the set of light sources 120 that are not within the subset are not illuminated). In a specific example, each light source within the sub-region of the set of light sources 120 can correspond to a pixel of a cropped image (e.g., a portion of the image that the user is viewing).

S400 can optionally include controlling the light engine 100 based on one or more user inputs (e.g., zoom selection, brightness selection, etc.). In an example, when the content includes an image, the light engine 100 can be controlled based on a cropped portion of the image, where the cropped portion of the image is determined based on a zoom selection.

However, the patterned light can be otherwise produced.

The method can optionally include directing the patterned light towards a target (e.g., the retina, the retinal implant 600, the target location, etc.) using one or more optical components. For example, the combiner lens 200 can be used to direct the patterned light output by the light engine 100 towards the retina.

The method can optionally include adjusting (e.g., calibrating) a position of one or more system components. For example, the method can include: measuring an inter-pupillary distance of the user, and adjusting a position of all or a portion of the light engine 100 (e.g., the set of light sources 120, the display module 140, etc.) and/or the combiner lens 200 based on the inter-pupillary distance of the user. In a specific example, the position of all or a portion of the light engine 100 (e.g., the set of light sources 120, the display module 140, etc.) and/or the combiner lens 200 can be adjusted within or on a glasses frame.

All or portions of the method can optionally be iteratively performed to update the projected light in real time with measurements received from the external sensor 300 and/or the eye sensor 400. For example, the method can include determining an updated target location (e.g., an update location of a retinal feature) based on an updated set of measurements from the eye sensor 400, selecting an updated sub-region of the set of light sources 120 based on the updated target location; and selectively illuminating at least a portion of the updated sub-region of the set of light sources 120 (e.g., wherein each light source in the array of light sources located outside the updated sub-region of the array of light sources is not illuminated).

However, the method can be otherwise performed.

### 5. Specific Examples

A numbered list of specific examples of the technology described herein are provided below. A person of skill in the art will recognize that the scope of the technology is not limited to and/or by these specific examples.

Specific Example 1. A system, comprising: a wearable projector system, comprising: a light engine comprising: an array of light sources configured to emit light; and a spatial light modulator configured to modulate the light to output patterned light; a combiner lens configured to direct the patterned light towards a retina of a user; an external sensor configured to sample an image; an eye sensor configured to sample a set of measurements of an eye feature; and a processing system configured to: track a location of the eye feature based on the set of measurements of the eye feature; control the spatial light modulator based on the image; determine a sub-region of the array of light sources based on the location of the eye feature; and control the light engine to selectively illuminate the sub-region of the array of light sources.

Specific Example 2. The system of Specific Example 1, wherein the wearable projector system further comprises a glasses frame, wherein the light engine, the combiner lens, the external sensor, the eye sensor, and the processing system are coupled to the glasses frame.

Specific Example 3. The system of Specific Example 2, wherein a position of the combiner lens on the glasses frame is adjustable.

Specific Example 4. The system of Specific Example 3, wherein the position of the combiner lens on the glasses frame is determined based on an inter-pupillary distance of the user.

Specific Example 5. The system of any of Specific Examples 1-4, wherein the array of light sources comprises an array of vertical-cavity surface-emitting lasers.

Specific Example 6. The system of any of Specific Examples 1-5, wherein the combiner lens is configured to project the patterned light onto a retinal implant, wherein the retinal implant is configured to generate electrical signals in response to receiving the patterned light.

Specific Example 7. The system of Specific Example 6, wherein the eye feature comprises a feature of the retinal implant.

Specific Example 8. The system of Specific Example 7, wherein the eye sensor is configured to sample the set of measurements of the feature of the retinal implant using infrared light.

Specific Example 9. The system of any of Specific Examples 1-8, wherein the spatial light modulator comprises a digital micromirror device.

Specific Example 10. The system of Specific Example 9, wherein controlling the spatial light modulator based on the image comprises determining an orientation of each mirror of the digital micromirror device based on the image, wherein a subset of the mirrors of the digital micromirror device are illuminated by the sub-region of the array of light sources.

Specific Example 11. A method, comprising: using an eye sensor, sampling a set of measurements of a retina of an eye of a user; tracking a location of a retinal feature, based on the set of measurements of the retina; selecting a sub-region of an array of light sources based on the location of the retinal feature; and producing patterned light, wherein the patterned light encodes content, wherein producing the patterned light comprises selectively illuminating at least a portion of the sub-region of the array of light sources, wherein each light source in the array of light sources located outside the sub-region of the array of light sources is not illuminated; and using a combiner lens, directing the patterned light towards the retina.

Specific Example 12. The method of Specific Example 11, further comprising controlling a spatial light modulator based on the content, wherein each light source within the sub-region of the array of light sources is illuminated, wherein the sub-region of the array of light sources projects unpatterned light onto a corresponding sub-region of the spatial light modulator, wherein the corresponding sub-region of the spatial light modulator modulates the unpatterned light to output the patterned light.

Specific Example 13. The method of Specific Example 12, further comprising: receiving a user input comprising a zoom selection; and controlling the spatial light modulator based on the content and the zoom selection.

Specific Example 14. The method of any of Specific Examples 12-13, wherein the spatial light modulator comprises a digital micromirror device, wherein controlling the spatial light modulator based on the image comprises determining an orientation of each mirror of the digital micromirror device based on the image, wherein the corresponding sub-region of the spatial light modulator comprises a subset of the mirrors of the digital micromirror device.

Specific Example 15. The method of any of Specific Examples 11-14, wherein producing the patterned light comprises: determining a subset of the sub-region of the array of light sources based on the content, and illuminating the subset of the sub-region of the array of light sources to produce the patterned light.

Specific Example 16. The method of any of Specific Examples 11-15, wherein the retinal feature comprises a feature of a retinal implant.

Specific Example 17. The method of any of Specific Examples 11-16, further comprising: determining an updated location of the retinal feature; selecting an updated sub-region of the array of light sources based on the updated location of the retinal feature; and selectively illuminating at least a portion of the updated sub-region of the array of light sources, wherein each light source in the array of light sources located outside the updated sub-region of the array of light sources is not illuminated.

Specific Example 18. The method of any of Specific Examples 11-17, wherein the wearable projector system further comprises a glasses frame, wherein the array of light sources and the combiner lens are coupled to the glasses frame, the method further comprising: measuring an inter-pupillary distance of the user; and adjusting a position of the combiner lens on the glasses frame based on the inter-pupillary distance of the user.

Specific Example 19. The method of Specific Example 18, the method further comprising adjusting the position of the array of light sources on the glasses frame based on the inter-pupillary distance of the user.

Specific Example 20. The method of any of Specific Examples 11-19, further comprising: receiving a user input comprising a brightness selection; and controlling a power of the sub-region of the array of light sources based on the brightness selection.

All references cited herein are incorporated by reference in their entirety, except to the extent that the incorporated material is inconsistent with the express disclosure herein, in which case the language in this disclosure controls.

As used herein, “substantially” or other words of approximation can be within a predetermined error threshold or tolerance of a metric, component, or other reference, and/or be otherwise interpreted.

Optional elements, which can be included in some variants but not others, are indicated in broken line in the figures.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A system, comprising:
a wearable projector system, comprising:
a light engine comprising:
an array of light sources configured to emit light; and
a spatial light modulator configured to modulate the light to output patterned light;
a combiner lens configured to direct the patterned light towards a retina of a user;
an external sensor configured to sample an image;
an eye sensor configured to sample a set of measurements of an eye feature; and
a processing system configured to:
track a location of the eye feature based on the set of measurements of the eye feature;
control the spatial light modulator based on the image;
determine a sub-region of the array of light sources based on the location of the eye feature; and
control the light engine to selectively illuminate the sub-region of the array of light sources.

2. The system of claim 1, wherein the wearable projector system further comprises a glasses frame, wherein the light engine, the combiner lens, the external sensor, the eye sensor, and the processing system are coupled to the glasses frame.

3. The system of claim 2, wherein a position of the combiner lens on the glasses frame is adjustable.

4. The system of claim 3, wherein the position of the combiner lens on the glasses frame is determined based on an inter-pupillary distance of the user.

5. The system of claim 1, wherein the array of light sources comprises an array of vertical-cavity surface-emitting lasers.

6. The system of claim 1, wherein the combiner lens is configured to project the patterned light onto a retinal implant, wherein the retinal implant is configured to generate electrical signals in response to receiving the patterned light.

7. The system of claim 6, wherein the eye feature comprises a feature of the retinal implant.

8. The system of claim 7, wherein the eye sensor is configured to sample the set of measurements of the feature of the retinal implant using infrared light.

9. The system of claim 1, wherein the spatial light modulator comprises a digital micromirror device.

10. The system of claim 9, wherein controlling the spatial light modulator based on the image comprises determining an orientation of each mirror of the digital micromirror device based on the image, wherein a subset of the mirrors of the digital micromirror device are illuminated by the sub-region of the array of light sources.

11. A method, comprising:
using an eye sensor, sampling a set of measurements of a retina of an eye of a user;
tracking a location of a retinal feature, based on the set of measurements of the retina;
selecting a sub-region of an array of light sources based on the location of the retinal feature; and
producing patterned light, wherein the patterned light encodes content, wherein producing the patterned light comprises selectively illuminating at least a portion of the sub-region of the array of light sources, wherein each light source in the array of light sources located outside the sub-region of the array of light sources is not illuminated; and
using a combiner lens, directing the patterned light towards the retina.

12. The method of claim 11, further comprising controlling a spatial light modulator based on the content, wherein each light source within the sub-region of the array of light sources is illuminated, wherein the sub-region of the array of light sources projects unpatterned light onto a corresponding sub-region of the spatial light modulator, wherein the corresponding sub-region of the spatial light modulator modulates the unpatterned light to output the patterned light.

13. The method of claim 12, further comprising:
receiving a user input comprising a zoom selection; and
controlling the spatial light modulator based on the content and the zoom selection.

14. The method of claim 12, wherein the spatial light modulator comprises a digital micromirror device, wherein controlling the spatial light modulator based on the image comprises determining an orientation of each mirror of the digital micromirror device based on the image, wherein the corresponding sub-region of the spatial light modulator comprises a subset of the mirrors of the digital micromirror device.

15. The method of claim 11, wherein producing the patterned light comprises:
determining a subset of the sub-region of the array of light sources based on the content, and illuminating the subset of the sub-region of the array of light sources to produce the patterned light.

16. The method of claim 11, wherein the retinal feature comprises a feature of a retinal implant.

17. The method of claim 11, further comprising:
determining an updated location of the retinal feature;
selecting a updated sub-region of the array of light sources based on the updated location of the retinal feature; and
selectively illuminating at least a portion of the updated sub-region of the array of light sources, wherein each light source in the array of light sources located outside the updated sub-region of the array of light sources is not illuminated.

18. The method of claim 11, wherein the wearable projector system further comprises a glasses frame, wherein the array of light sources and the combiner lens are coupled to the glasses frame, the method further comprising:
measuring an inter-pupillary distance of the user; and
adjusting a position of the combiner lens on the glasses frame based on the inter-pupillary distance of the user.

19. The method of claim 18, the method further comprising adjusting the position of the array of light sources on the glasses frame based on the inter-pupillary distance of the user.

20. The method of claim 11, further comprising:
receiving a user input comprising a brightness selection; and
controlling a power of the sub-region of the array of light sources based on the brightness selection.
